(19) United States
(12) Patent Application Publication
(10) Pub. No.: US 2024/0374336 A1
(43) Pub. Date: Nov. 14, 2024

(54) TAMPER PROOF CABLE LOCK

(71) Applicant: Steven Joseph Jaworski, East Yaphank, NY (US)

(72) Inventor: Steven Joseph Jaworski, East Yaphank, NY (US)

(21) Appl. No.: 18/657,309

(22) Filed: May 7, 2024

Related U.S. Application Data
(60) Provisional application No. 63/464,837, filed on May 8, 2023.

(51) Int. Cl. A61B 50/20; A61B 50/00; A61M 25/02

(52) U.S. Cl. CPC A61B 50/20; A61M 2025/024

(57) ABSTRACT

A cable lock includes a security rod defining a first end portion, a second end portion, and an extension portion. A first locking clasp is coupled with the first end portion of the security rod. The first locking clasp includes a first clasp component coupled with the first end portion of the security rod. A second clasp component is at least partially separable from the first clasp component. The first clasp component and/or the second clasp component defines a first orifice configured to receive a first device cable. A second locking clasp is coupled with the second end portion of the security rod. The second clasp component includes a second orifice configured to receive a second device cable. The security rod, the first locking clasp, and the second locking clasp are configured to prevent removal of the first device cable and the second device cable from a medical device.

### CROSS-REFERENCE TO RELATED APPLICATION

This application claims priority to U.S. Provisional Patent Application No. 63/464,837 filed on May 8, 2023, the disclosure of which is incorporated by reference herein in its entirety.

### FIELD

The present disclosure relates to a cable lock and, more particularly, to a tamper proof cable lock.

### BACKGROUND

Medical facilities, such as hospitals, commonly use roving medical devices and medical equipment that can be transported to various locations in the medical facility. For example, emergency departments triage and deliver medical care for a wide array of medical problems requiring different medical devices and equipment. Similarly, operating rooms in medical facilities perform a multitude of surgeries requiring, for example, different electronic monitors and electrical leads for the monitors. Roving medical devices, such as bedside monitors, defibrillators and mobile vital signs monitors may be attached to portable stands that allow the devices to be quickly moved in and out of various locations in the medical facility.

Each medical device may require a unique combination of one or more electrical cables or leads for the device to be powered and operate correctly. Medical devices may also require unique combinations of electrical cables to perform a desired combination of tasks based on the medical needs or issues of a particular patient. However, electrical cables are generally easily removable from the device with which they are associated. This results in a common and expensive problem of loss of electrical cables and device leads that may cost an individual medical facility upwards of millions of dollars annually.

Another common and expensive problem leading to a loss of electrical cables and leads occurs when a particular cable fails while the cable may still be under warranty, and the cable is discarded by hospital staff without the cable being submitted to the manufacturer for replacement. In particular, device cables are frequently lost or discarded in high traffic areas of hospitals, such as emergency departments and operating rooms. In addition to creating waste and increasing operational costs for medical facilities, the loss of device cables also indirectly increases the cost of healthcare for patients. Lost device cables may also decrease the availability of functional medical equipment in medical facilities and therefore indirectly lead to increased patient wait times and decreased quality of care.

### SUMMARY

Provided in accordance with aspects of the present disclosure is a cable lock including a security rod defining a first end portion, a second end portion, and an extension portion extending between the first end portion and the second end portion. The security rod includes a rigid metal core and a pliable coating arranged on the rigid metal core along the extension portion of the security rod. A first locking clasp is coupled with the first end portion of the security rod. The first locking clasp includes a first clasp component and a first security rod connecting arm connecting the first clasp component with the security rod. The first locking clasp includes a second clasp component and a second security rod connecting arm connecting the second clasp component with the security rod. A first orifice is defined between the first clasp component and the second clasp component. The first orifice is configured to receive a first device cable. A second locking clasp is coupled with the second end portion of the security rod. The second locking clasp includes a third clasp component and a third security rod connecting arm connecting the third clasp component with the security rod. The second locking clasp includes a fourth clasp component and a fourth security rod connecting arm connecting the fourth clasp component with the security rod. A second orifice is defined between the third clasp component and the fourth clasp component. The second orifice is configured to receive a second device cable. The security rod prevents removal of the first device cable and the second device cable from a medical device.

In an aspect of the present disclosure, a first stop sleeve is coupled with the first end portion of the security rod. A first washer is arranged between the first stop sleeve and the first security rod connecting arm of the first locking clap. A second washer is arranged between the second security rod connecting arm and the pliable coating of the security rod. A third washer is arranged between the pliable coating of the security rod and the third security rod connecting arm of the second clasp component. A second stop sleeve is coupled with the second end portion of the security rod. A fourth washer is arranged between the fourth security rod connecting arm of the second clasp component and the second stop sleeve.

In an aspect of the present disclosure, at least one of the first stop sleeve or the second stop sleeve is in direct metal to metal contact with the rigid metal core of the security rod. At least one of the first washer, the second washer, the third washer, or the fourth washer is in rotatable contact with the rigid metal core of the security rod.

In an aspect of the present disclosure, at least one of the first security rod connecting arm, the second security rod connecting arm, the third security rod connecting arm, or the fourth security rod connecting arm is in rotatable contact with the rigid metal core of the security rod.

In an aspect of the present disclosure, an adhesive is arranged between the pliable coating and the rigid metal core of the security rod. The adhesive secures the pliable coating to the rigid metal core.

In an aspect of the present disclosure, the extension portion of the security rod defines a first vertical portion, a second vertical portion, and a horizontal portion connecting the first vertical portion with the second vertical portion. The first vertical portion, the second vertical portion, and the horizontal portion are arranged to allow the medical device to be positioned in a support sleeve.

In an aspect of the present disclosure, a first curved portion of the security rod connects the first vertical portion with the horizontal portion. A second curved portion of the security rod connects the second vertical portion with the horizontal portion.

In an aspect of the present disclosure, the first security rod connecting arm, the second security rod connecting arm, the third security rod connecting arm, and the fourth security rod connecting arm extend from the security rod to align the first clasp component and the second clasp component with an upper surface of the medical device and to align the third clasp component and the fourth clasp component with a bottom surface of the medical device.

In an aspect of the present disclosure, the first clasp component, the second clasp component, the third clasp component, and the fourth clasp component extend in a plane offset from a plane in which the security rod extends.

In an aspect of the present disclosure, at least one of the first clasp component, the second clasp component, the third clasp component, or the fourth clasp component includes a strip extending along a length of the first clasp component, the second clasp component, the third clasp component, or the fourth clasp component. The strip is configured to contact the first device cable or the second device cable of the medical device to prevent movement of the first device cable or the second device cable between the first clasp component and the second clasp component or between the third clasp component and the fourth clasp component.

In an aspect of the present disclosure, an indent is formed in the first clasp component, the second clasp component, the third clasp component, or the fourth clasp component. The strip is at least partially arranged in the indent.

In an aspect of the present disclosure, the strip protrudes from the indent.

In an aspect of the present disclosure, a screw removably couples the first clasp component to the second clasp component or the third clasp component to the fourth clasp component.

In an aspect of the present disclosure, the screw includes a counter sunk head configured to be disposed in the first clasp component, the second clasp component, the third clasp component, or the fourth clasp component.

In an aspect of the present disclosure, the rigid metal core of the security rod includes or is formed of stainless steel. The washers may also include or may be formed of stainless steel.

In an aspect of the present disclosure, the pliable coating of the security rod includes a shrink tube.

In an aspect of the present disclosure, the pliable coating arranged on the rigid metal core along the extension portion of the security rod forms a first lateral surface facing along an outer surface of the rigid metal core of the security rod, and a second lateral surface facing along the outer surface of the rigid metal core of the security rod.

In an aspect of the present disclosure, the first lateral surface is arranged to face a third lateral surface of the second security rod connecting arm. The second lateral surface is arranged to face a fourth lateral surface of the third security rod connecting arm.

Provided in accordance with aspects of the present disclosure is a cable lock including a security rod defining a first end portion, a second end portion, and an extension portion extending between the first end portion and the second end portion of the security rod. A first locking clasp is coupled with the first end portion of the security rod. The first locking clasp includes a first clasp component coupled with the first end portion of the security rod connecting arm. A second clasp component is at least partially separable from the first clasp component. The first clasp component and/or the second clasp component defines a first orifice configured to receive a first device cable. A second locking clasp is coupled with the second end portion of the security rod. The second clasp component includes a second orifice configured to receive a second device cable. The security rod, the first locking clasp, and the second locking clasp are configured to prevent removal of the first device cable and the second device cable from a medical device.

In an aspect of the present disclosure, a cuboidal segment is defined by the first end portion of the security rod. A cuboidal orifice is formed in the first clasp component of the first locking clasp. The cuboidal orifice is configured to receive the cuboidal segment to couple the first clasp component with the first end portion of the security rod. The cuboidal segment of the first end portion of the security rod is configured to prevent rotation of the first locking clasp.

In an aspect of the present disclosure, the extension portion of the security rod defines a cylindrical shape. A first cross-sectional area defined by the cuboidal segment of the first end portion of the security rod is smaller than a second cross-sectional area defined by the extension portion of the security rod.

In an aspect of the present disclosure, the extension portion of the security rod defines a cylindrical shape. A first diameter of the cuboidal segment of the first end portion of the security rod is smaller than a second diameter of the extension portion of the security rod.

In an aspect of the present disclosure, the security rod is a rigid rod configured to prevent movement of the first locking clasp or the second locking clasp with respect to the medical device.

In an aspect of the present disclosure, the security rod includes aluminum.

In an aspect of the present disclosure, the first locking clasp and the second locking clasp extend from the security rod to align the first clasp component and the second clasp component with an upper surface of the medical device and to align the second locking clasp with a bottom surface of the medical device. The first orifice of the first locking clasp and the second orifice of the second locking clasp each extend in a plane offset from a plane in which the security rod extends.

In an aspect of the present disclosure, a screw is configured to removably couple the first clasp component to the second clasp component. The screw includes a counter sunk head configured to be disposed in the first clasp component.

In an aspect of the present disclosure, a first screw is configured to couple the first clasp component with the security rod. The first screw is configured to be flush with a surface of the first clasp component facing away from the medical device. A second screw is configured to couple the second clasp component with the security rod. The second screw is configured to be flush with a surface of the second clasp component facing away from the medical device.

In an aspect of the present disclosure, the second end portion of the security rod defines a cuboidal segment configured to prevent rotation of the second locking clasp.

In an aspect of the present disclosure, the second clasp component includes a cradle configured to hold a connecting arm of the second device cable. The second orifice extends from the cradle.

In an aspect of the present disclosure, the cradle is configured to face the medical device. The second orifice extends in a direction perpendicular to a direction in which the cradle faces.

In an aspect of the present disclosure, the second clasp component includes a third clasp component including a first security rod connecting arm connecting the third clasp component with the security rod. A fourth clasp component includes a second security rod connecting arm connecting the fourth clasp component with the security rod. The second orifice is defined between the third clasp component and the fourth clasp component.

In an aspect of the present disclosure, a cuboidal segment is defined by the second end portion of the security rod. A cuboidal orifice is defined in the first security rod connecting arm. The cuboidal orifice is configured to receive the cuboidal segment to prevent rotation of the third clasp component. A cylindrical segment is defined by the second end portion of the security rod. A cylindrical orifice is defined in the second security rod connecting arm. The cylindrical orifice is configured to receive the cylindrical segment to allow rotation of the fourth clasp component with respect to the third clasp component.

In an aspect of the present disclosure, a washer is arranged about the cylindrical segment defined by the second end portion of the security rod. The washer is configured to receive a screw connecting the second locking clasp with the security rod.

### BRIEF DESCRIPTION OF THE DRAWINGS

Various aspects and features of the present disclosure are described hereinbelow with reference to the drawings wherein:

FIG. 1 is a front view of a cable lock according to aspects of the present disclosure;

FIG. 2 is a front view of the cable lock of FIG. 1 arranged about a medical device with first and second locking clasps arranged in open positions;

FIG. 3 is a front view of the cable lock of FIG. 1 arranged about the medical device with the first and second locking clasps arranged in closed positions and securing the device cables of the medical device to the medical device;

FIG. 4A is a perspective view of a locking clasp of the cable lock of FIG. 1 in a closed position;

FIG. 4B is a perspective view of the locking clasp of FIG. 4A in an open position;

FIG. 5A is a perspective view of another locking clasp of the cable lock of FIG. 1 in a closed position;

FIG. 5B is a perspective view of the locking clasp of FIG. 5A in an open position;

FIG. 6 is an enlarged view of the locking clasp of FIG. 5B illustrating the strip configured to prevent movement of the device cables of the medical device;

FIG. 7A is an enlarged view of the locking clasp of FIG. 5B with the device cables of the medical device arranged in the locking clasp;

FIG. 7B is an enlarged view of the locking clasp of FIG. 4B with a device cable of the medical device arranged in the locking clasp;

FIG. 8A is a side, perspective view of the cable lock of FIG. 1 coupled to the medical device;

FIG. 8B is a side view of the cable lock of FIG. 8A showing the arrangement of the locking clasps with respect to the top and bottom of the medical device;

FIG. 9A is a front view of the cable lock of FIG. 8A coupled to the medical device, with the medical device arranged in a support sleeve;

FIG. 9B is a side, perspective view of the cable lock of FIG. 9A with the medical device arranged in the support sleeve;

FIG. 10 is a front view of a cable lock according to aspects of the present disclosure;

FIG. 11A is a top, perspective view of the cable lock of FIG. 10;

FIG. 11B is a bottom, perspective view of the cable lock of FIG. 10;

FIG. 12A is an enlarged, perspective view of a first locking clasp of the cable lock of FIG. 10;

FIG. 12B is an enlarged, perspective view of an embodiment of a second locking clasp employable by the cable lock of FIG. 10;

FIG. 13A is an enlarged view of the first locking clasp of FIG. 12A in a partially separated arrangement;

FIG. 13B is an enlarged view of the first locking clasp of FIG. 12A in a completely separated arrangement;

FIG. 14A is an enlarged view of the first locking clasp of FIG. 12A separated from a first end portion of a security rod;

FIG. 14B is an enlarged view of an embodiment of a second end portion of a security rod configured to receive an embodiment of a second lock clasp;

FIG. 15A is a perspective view of another embodiment of a second locking clasp employable by the cable lock of FIG. 10; and

FIG. 15B is a plan view of the second locking clasp of FIG. 15A separated from a security rod.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

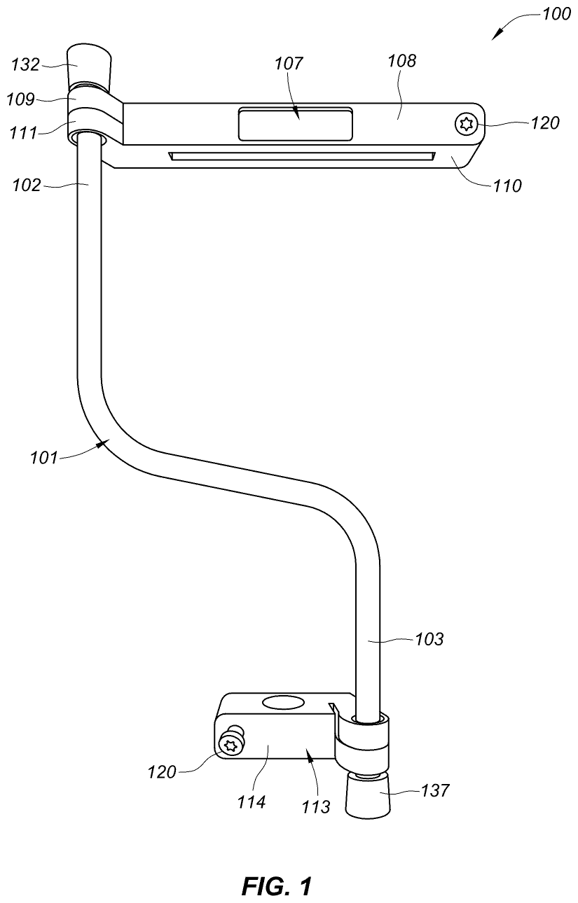

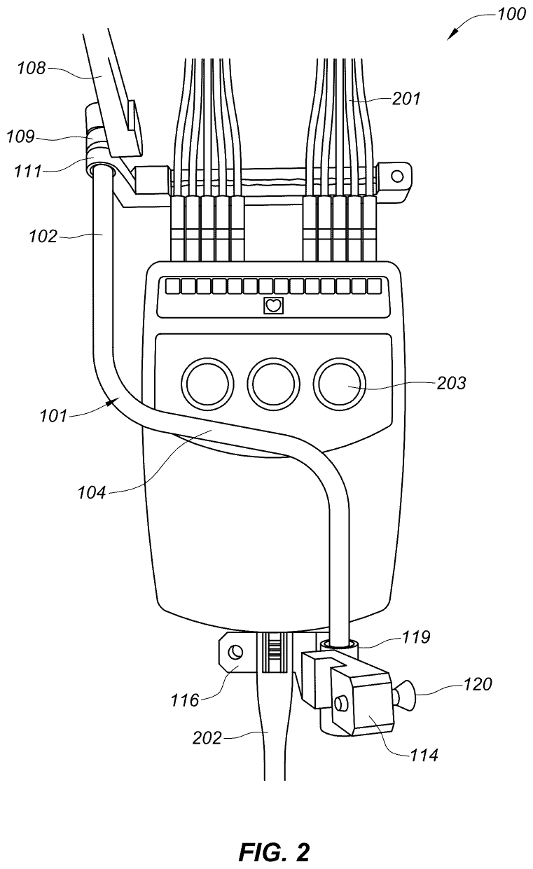

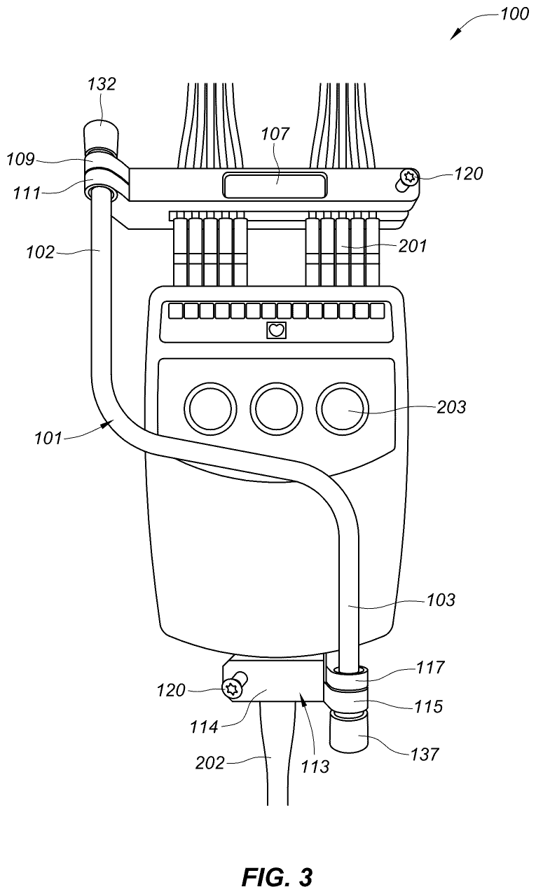

Referring to FIGS. 1-3, a cable lock 100 includes a security rod 101 defining a first end portion 102, a second end portion 103, and an extension portion 104 extending between the first end portion 102 and the second end portion 103. The security rod 101 may include a rigid metal core and a pliable coating arranged on the rigid metal core along the extension portion 104 of the security rod 101.

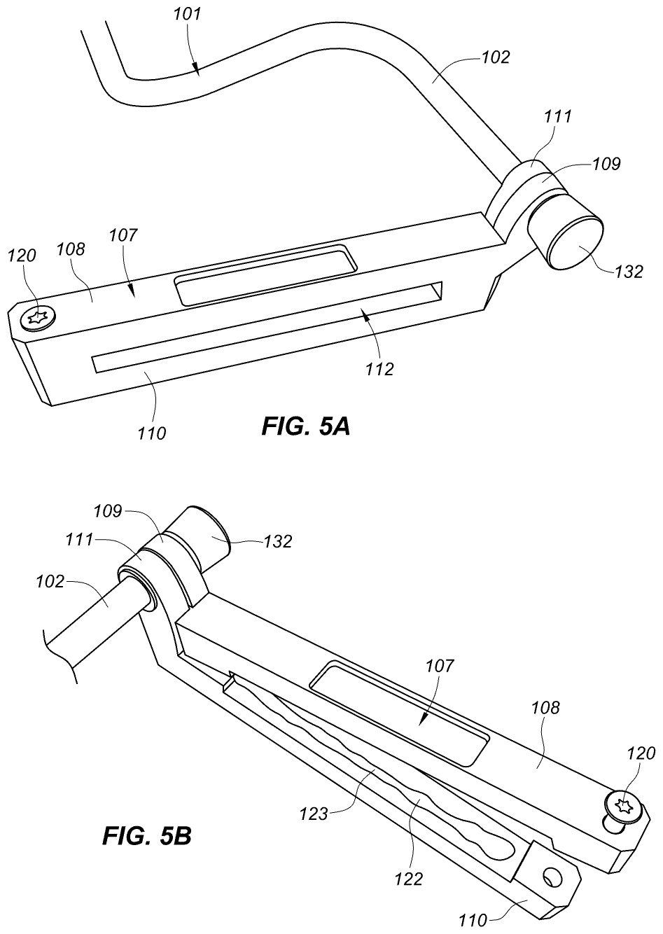

A first locking clasp 107 is coupled with the first end portion 102 of the security rod 101. The first locking clasp 107 includes a first clasp component 108 and a first security rod connecting arm 109 connecting the first clasp component 108 with the security rod 101. The first locking clasp 107 includes a second clasp component 110 and a second security rod connecting arm 111 connecting the second clasp component 110 with the security rod 101. A first orifice 112 (see, e.g., FIG. 5A) is defined between the first clasp component 108 and the second clasp component 110. The first orifice 112 is configured to receive a first device cable 201.

A second locking clasp 113 is coupled with the second end portion 103 of the security rod 101. The second locking clasp 113 includes a third clasp component 114 and a third security rod connecting arm 115 connecting the third clasp component 114 with the security rod 101. The second locking clasp 113 includes a fourth clasp component 116 and a fourth security rod connecting arm 117 connecting the fourth clasp component 116 with the security rod 101. A second orifice 118 is defined between the third clasp component 114 and the fourth clasp component 116. The second orifice 118 is configured to receive a second device cable 202. The security rod 101 prevents removal of the first device cable 201 and the second device cable 202 from a medical device 203 (e.g., by preventing upward or downward movement of the device cables). The security rod 101 has a length and arrangement that positions the first and second locking clasps 107 and 113 immediately adjacent top and bottom surfaces of the medical device 203, respectively, such that the cables of the medical device 203 cannot be advanced in an upward or downward direction, or otherwise removed from the medical device 203. Thus, the cables cannot be removed from the medical device 203 when the cable lock 100 is in use. As an example, the security rod 101 might not be connected directly to the medical device 203 because the locking clasps utilize the attachment between the device cables and the medical device 203 to hold the cable lock 100 about the medical device 203 while preventing removal of the device cables. This provides the unexpected benefit of avoiding the need for additional attachment components directly to the medical device and reduces manufacturing costs.

In an aspect of the present disclosure, the rigid metal core of the security rod 101 includes or is formed of stainless steel. The washers 119 may also include or may be formed of stainless steel. While a metal core rod, or metal washers may be employed, other rigid materials may similarly be employed (e.g., high density/rigid plastics or polymers).

In an aspect of the present disclosure, the pliable coating of the security rod 101 includes a shrink tube, such as a moisture-seal heat-shrink semi-rigid tubing material. The pliable coating may include or may be formed of plastic, rubber, or another similar flexible material. For example, the pliable coating may include or may be formed of polyolefin plastic. The pliable coating may be shrink-sealed about the extending portion of the rigid metal core of the security rod 101, while leaving metal exposed at the end portions of the security rod 101. An adhesive arranged on the inner surface of the pliable coating may adhere the pliable coating to the rigid metal core. The adhesive may be heat-activated to increase bonding strength between the pliable coating and the rigid metal core.

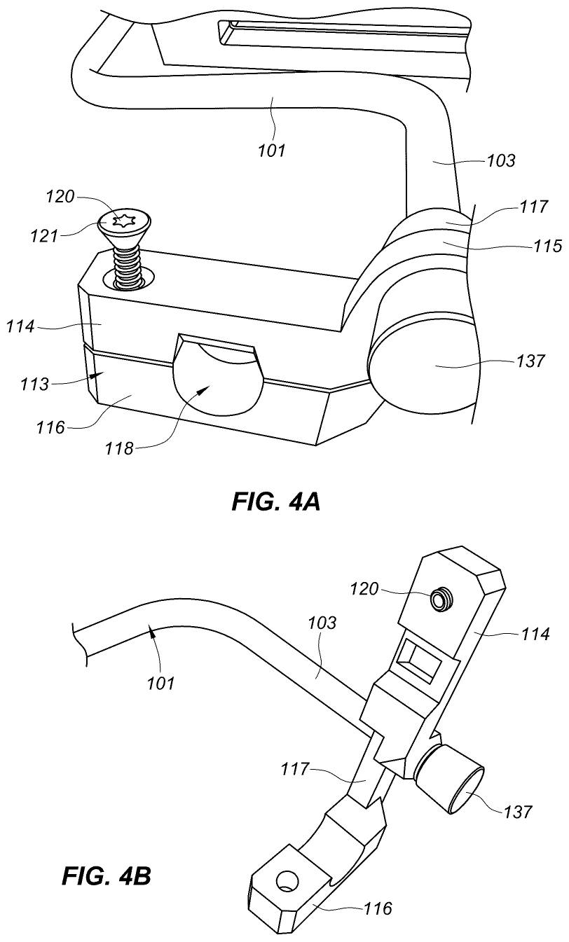

Referring particularly to FIGS. 4A to 5B, a screw 120 removably couples the first clasp component 108 to the second clasp component 110 or the third clasp component 114 to the fourth clasp component 116. The screw 120 may include a counter sunk head 121 configured to be disposed in the first clasp component 108, the second clasp component 110, the third clasp component 114, and/or the fourth clasp component 116. The clasp components may be configured to pivot away from each other about the rigid metal core of the security rod 101.

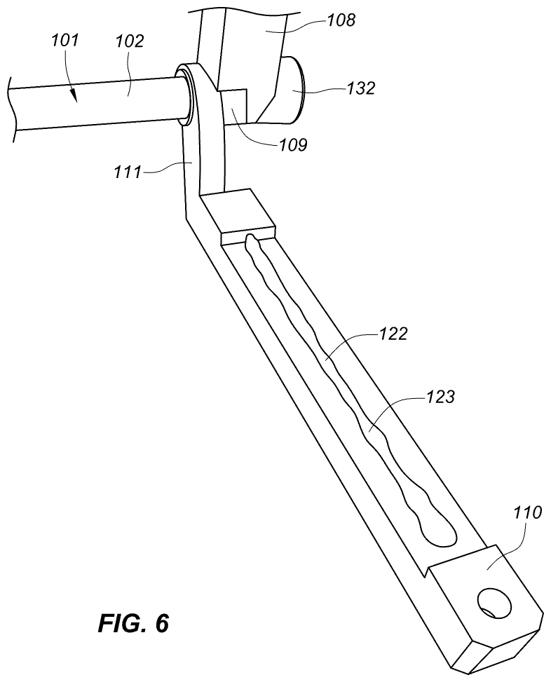

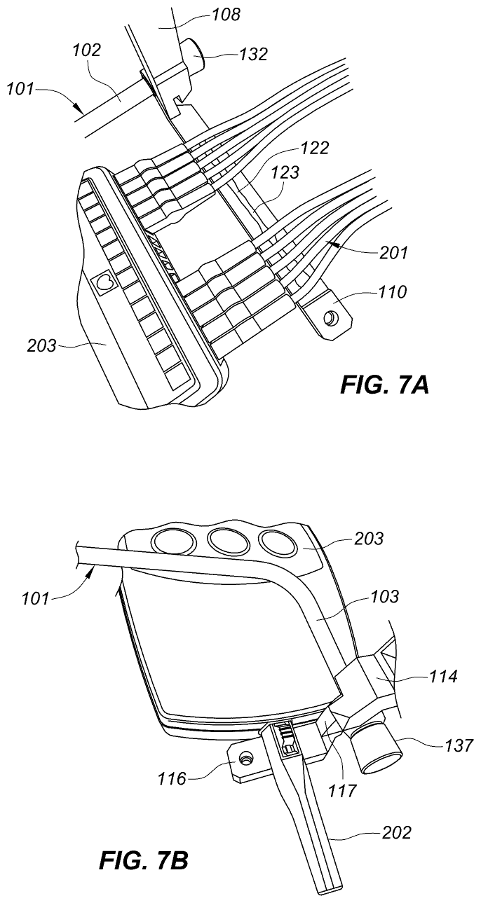

Referring particularly to FIG. 5B to 7B, at least one of the first clasp component 108, the second clasp component 110, the third clasp component 114, or the fourth clasp component 116 includes a strip 122 extending along a length of the first clasp component 108, the second clasp component 110, the third clasp component 114, or the fourth clasp component 116. The strip 122 is configured to contact the first device cable 201 or the second device cable 202 of the medical device 203 to prevent movement of the first device cable 201 or the second device cable 202 between the first clasp component 108 and the second clasp component 110 or between the third clasp component 114 and the fourth clasp component 116. As an example, a separate strip may be formed on each clasp component.

As an example, the strip 122 may include or may be formed of glue (e.g., glue dispensed by a hot glue gun), rubber, liquid rubber, plastic, or any pliable and/or tacky material configured to grip an outer coating of medical device cables to prevent movement (e.g., to prevent lateral or longitudinal movement) of medical device cables held by the locking clasps described herein.

In an aspect of the present disclosure, an indent 123 is formed in the first clasp component 108, the second clasp component 110, the third clasp component 114, or the fourth clasp component 116. The strip 122 is at least partially arranged in the indent 123. As an example, the strip 122 may at least partially protrude from the indent 123.

Referring particularly to FIGS. 1-3, and 8A, the pliable coating arranged on the rigid metal core along the extension portion 104 of the security rod 101 forms a first lateral surface facing along an outer surface of the rigid metal core of the security rod 101, and a second lateral surface facing along the outer surface of the rigid metal core of the security rod 101. That is, the pliable coating increases a thickness of the security rod 101 (along the extending portion) with respect to the rigid metal core and creates lateral facing surfaces at the opposite ends of the pliable coating. The washers 119 or the security rod connecting arms (e.g., connecting arms 109, 111, 115, and/or 117) may contact the lateral surfaces to prevent longitudinal movement of the locking clasps along the security rod 101. That is, a thickness of apertures formed in a center of the washers 119 and/or the security rod connecting arms may be substantially the same as a thickness of the rigid metal portion of the security rod 101, and the thickness of the portion of the security rod 101 having the pliable coating may be thicker than the apertures formed in the center of the washers 119 and/or the security rod connecting arms. The security rod connecting arms are functionally sandwiched between a stop sleeve 137 at one, and a lateral surface of the pliable coating at the opposite side, and the washers 119 may be arranged between the stop sleeve 137 and a security rod connecting arm and between the other security rod connecting arm and the lateral surface of the pliable coating. The washers 119 allow for easier rotation of the security rod connecting arms about the rigid metal core of the security rod 101.

For example, the first lateral surface is arranged to face a third lateral surface of the second security rod connecting arm 111. The second lateral surface is arranged to face a fourth lateral surface of the third security rod connecting arm 115.

Referring particularly to FIGS. 1 to 3 again, a first stop sleeve 132 is coupled with the first end portion 102 of the security rod 101. A first washer is arranged between the first stop sleeve 132 and the first security rod connecting arm 109 of the first locking clasp 107. A second washer is arranged between the second security rod connecting arm 111 and the pliable coating of the security rod 101. A third washer is arranged between the pliable coating of the security rod 101 and the fourth security rod connecting arm 117 of the second locking clasp 113. A second stop sleeve 137 is coupled with the second end portion 103 of the security rod 101. A fourth washer is arranged between the third security rod connecting arm 115 of the second locking clasp 113 and the second stop sleeve 137. The first and second stop sleeves 132 and 137 may each include a pliable cap.

In an aspect of the present disclosure, at least one of the first stop sleeve 132 or the second stop sleeve 137 is in direct metal to metal contact with the rigid metal core of the security rod 101. At least one of the first washer, the second washer, the third washer, or the fourth washer (see, e.g., washer 119) is in rotatable contact with the rigid metal core of the security rod 101.

In an aspect of the present disclosure, at least one of the first security rod connecting arm 109, the second security rod connecting arm 111, the third security rod connecting arm 115, or the fourth security rod connecting arm 117 is in rotatable contact with the rigid metal core of the security rod 101.

In an aspect of the present disclosure, an adhesive is arranged between the pliable coating and the rigid metal core of the security rod 101. The adhesive secures the pliable coating to the rigid metal core.

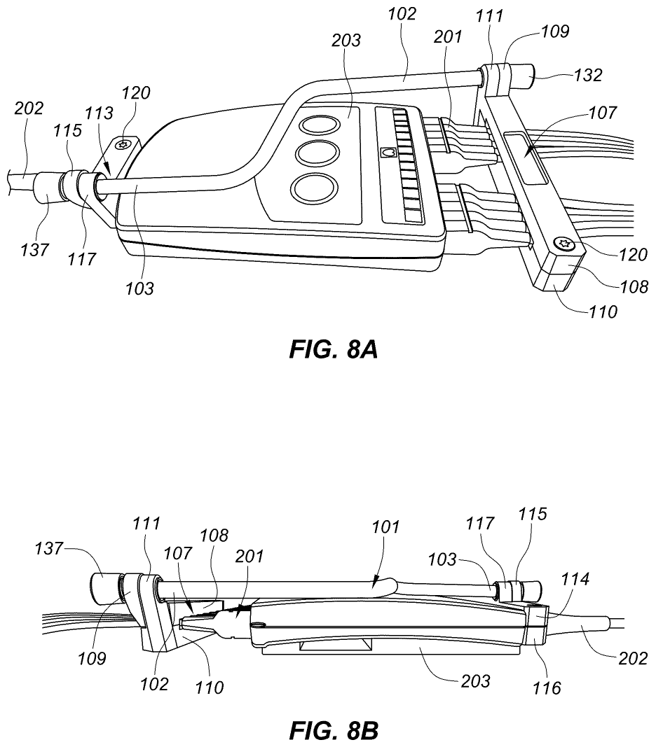

Referring particularly to FIG. 8B, the first security rod connecting arm 109, the second security rod connecting arm 111, the third security rod connecting arm 115, and the fourth security rod connecting arm 117 extend from the security rod 101 to align the first clasp component 108 and the second clasp component 110 with an upper surface of the medical device 203 and to align the third clasp component 114 and the fourth clasp component 116 with a bottom surface of the medical device 203.

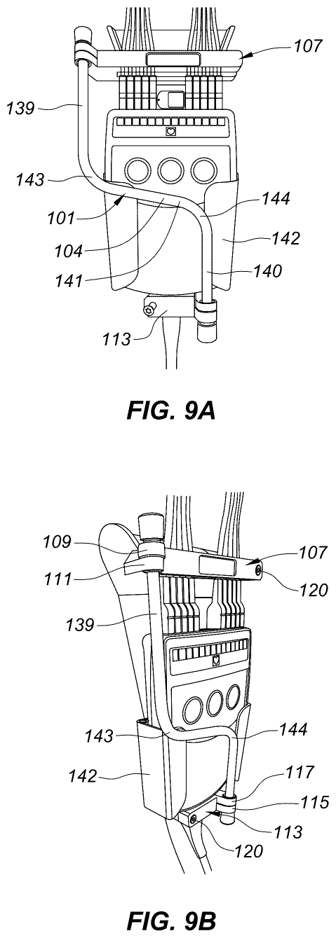

In an aspect of the present disclosure, the first clasp component 108, the second clasp component 110, the third clasp component 114, and the fourth clasp component 116 extend in a plane offset from a plane in which the security rod 101 extends. The security rod 101 extends along the front surface of the medical device 203. As illustrated in FIGS. 9A and 9B, discussed in more detail below, arranging the security rod 101 along the front surface of the medical device 203 allows the medical device 203 to be positioned in a holding sleeve without being disrupted by the security rod 101 or any of the components operably coupled with the security rod 101.

Referring particularly to FIGS. 9A and 9B, the extension portion 104 of the security rod 101 defines a first vertical portion 139, a second vertical portion 140, and a horizontal portion 141 connecting the first vertical portion 139 with the second vertical portion 140. The first vertical portion 139, the second vertical portion 140, and the horizontal portion 141 are arranged to allow the medical device 203 to be positioned in a support sleeve 142.

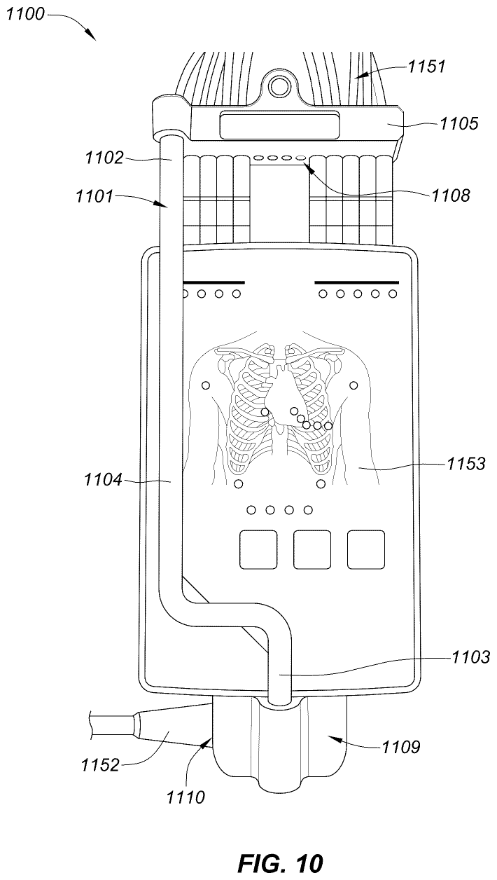

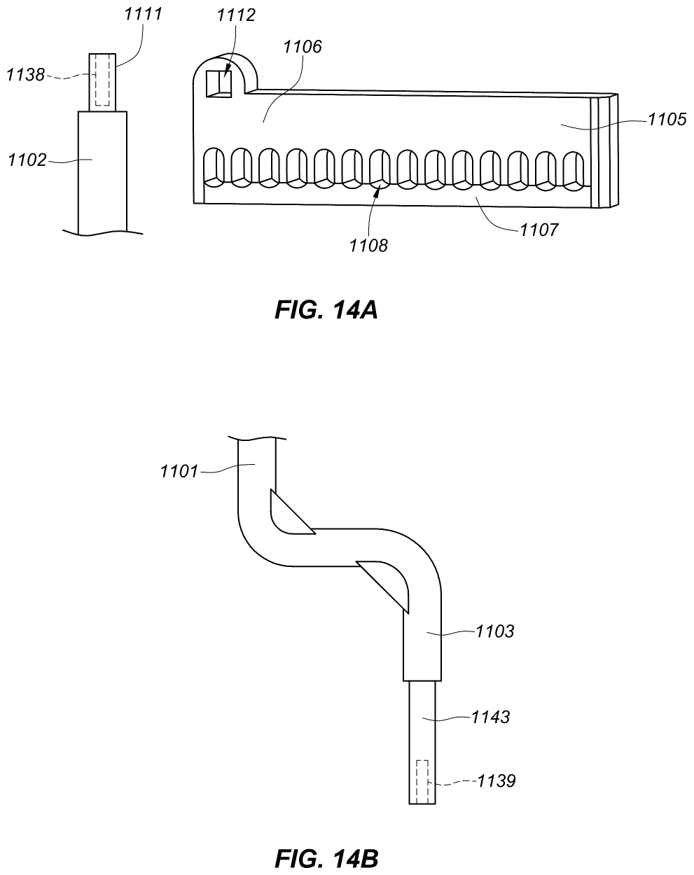

In an aspect of the present disclosure, a first curved portion 143 of the security rod 101 connects the first vertical portion 139 with the horizontal portion 141. A second curved portion 144 of the security rod 101 connects the second vertical portion 140 with the horizontal portion 141. Reinforcing members (see, e.g., FIGS. 10 and 14B) may be employed to reinforce the first and second curved portions 143 and 144 to prevent bending of the security rod 101.

In use, the first point of contact of the cable lock 100 is the locking clasp 113 at the bottom of the medical device 203 that attaches around the molded connector below the medical device 203, disabling the function of the quick release of the molded connector, thus securing the device cable into the medical device 203 (e.g., a device module). The locking clasp 113 may include two plates (see, e.g., clasp components 114 and 116 in FIG. 4A), which when placed on top of each other form a unique cavity (see, e.g., orifice 118) in the center that mirrors the dimensions of the product it secures. Via the top plate a specialized security screw (e.g., a tamper proof screw) is inserted into a countersunk hole that runs through the side of both plates and fastens into a nut that is pressed into the lower plate or threads directly into the lower plate.

The plates of the locking clasp 113 may define an offset hinge, created by holes strategically placed in the plates for which the metal rod is inserted through and acts as the pivot point. This assembly is secured using an aluminum crimp stop sleeve 137 that may include a plastic cap (e.g., a plastic cap secured in place by an adhesive), to cover the metal termination points. By loosening the security screw (e.g., from the impregnated nut in the lower plate), the top plate and lower plate will swivel open allowing the user to access and exchange components, and by repeating the steps in reverse the user can lock the clasp over the product they wish to secure.

The security rod 101 which acts as the pivot point for the hinge in the clasps, may be formed of or may include a solid stainless-steel rod, coated in a protective rubberized shrink tubing. The security rod 101 can be bent into shape to follow the contours of the top of the medical module. The structure of the security rod 101 does not flex and cannot be manipulated.

The second point of contact of the cable lock 100 is the locking clasp 107 at the top of the medical device module that attaches around multiple round wires (e.g., upwards of 16 wires). The upper locking clasp 107 may include two plates (see, e.g., clasp components 108 and 110), which when placed on top of each other forms an orifice (or multiple orifices-see, e.g., orifice 112 in FIG. 5A) that is the same thickness of the product wiring/cables it secures. Within the lower plate runs a small cavity in which a strip of rubberized material can be placed to act as a soft yet rigid sponge that keeps the clasp from flexing by creating a tight contact with the products it is clamping against. Via the top plate a specialized security screw is inserted into a countersunk hole that runs through the right side of both plates and may fasten into a nut that is pressed into the lower plate or may be threaded directly into the lower plate. One side of the plate has an offset hinge, created by holes strategically placed in the plates for which the security rod 101 is inserted through and acts as the pivot point. This assembly is secured using an aluminum crimp stop sleeve 132 that may include a plastic cap (e.g., a cap that is secured in place by an adhesive) to cover the metal termination points. By loosening the security screw from the impregnated nut in the lower plate or from the lower plate, the top plate and lower plate can swivel open allowing the user to access and exchange components (e.g., wires, leads, or cables), and by repeating the steps in reverse the user can lock the clasp over the product they wish to secure.

Various arrangements of locking clasps are described in U.S. Pat. No. 11,098,502, the entire contents of which are hereby incorporated by reference.

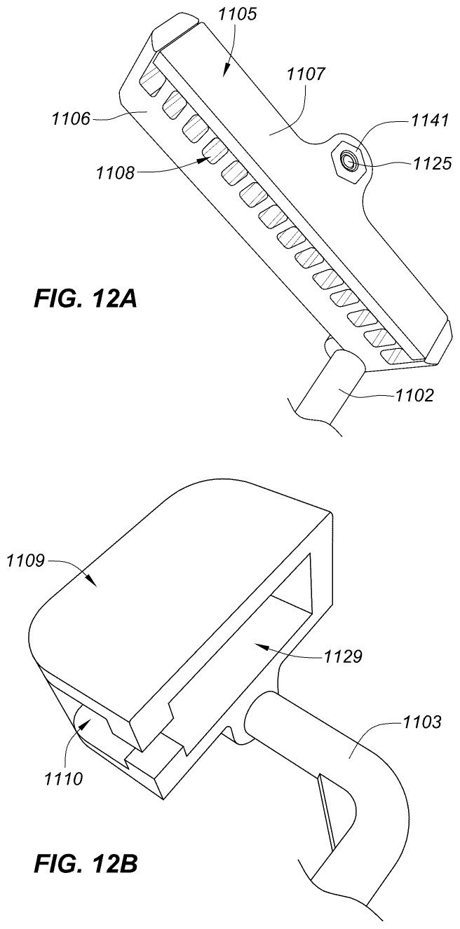

Referring to FIGS. 10, 11A, 11B, 12A, and 12B, a cable lock 1100 includes a security rod 1101 defining a first end portion 1102, a second end portion 1103, and an extension portion 1104 extending between the first end portion 1101 and the second end portion 1103 of the security rod 1101. A first locking clasp 1105 is coupled with the first end portion 1102 of the security rod 1101. The first locking clasp 1105 includes a first clasp component 1106 coupled with the first end portion 1102 of the security rod 1101. A second clasp component 1107 is at least partially separable from the first clasp component 1106. The first clasp component 1106 and/or the second clasp component 1107 define(s) a first orifice 1108 (see, e.g., FIG. 12A) configured to receive a first device cable 1151. A plurality of orifices may be defined in the first locking clasp 1105 to secure a plurality of device cables, and each of the individual device cables may be separated from each other by sidewalls defined by the first locking clasp 1105.

A second locking clasp 1109 is coupled with the second end portion 1103 of the security rod 1101. The second locking clasp 1109 includes a second orifice 1110 (see, e.g., FIG. 12B) configured to receive a second device cable 1152. The security rod 1101, the first locking clasp 1105, and the second locking clasp 1109 are configured to prevent removal of the first device cable(s) 1151 and the second device cable 1152 from a medical device 1153.

In use, the cable lock 1100 prevents device cables from being removed from a medical device 1153, such as an EKG monitor, by preventing the device cables from being pulled out of the connecting ports of the medical device. The arrangement of the first and second locking clasps 1105 and 1109 prevents rotation of the first and second locking clasps with respect to the security rod 1101. Preventing rotational motion prevents tampering with the cable lock 1100 and the device cables, thus preventing, or substantially mitigating an occurrence, of unwanted removal of device cables from the medical device 1153. As an example, the security rod 1101 might not be connected directly to the medical device 1153 because the locking clasps utilize the attachment between the device cables and the medical device 1153 to hold the cable lock 1100 about the medical device 1153 while preventing removal of the device cables. This provides the unexpected benefit of avoiding the need for additional attachment components directly to the medical device 1153 and reduces manufacturing costs.

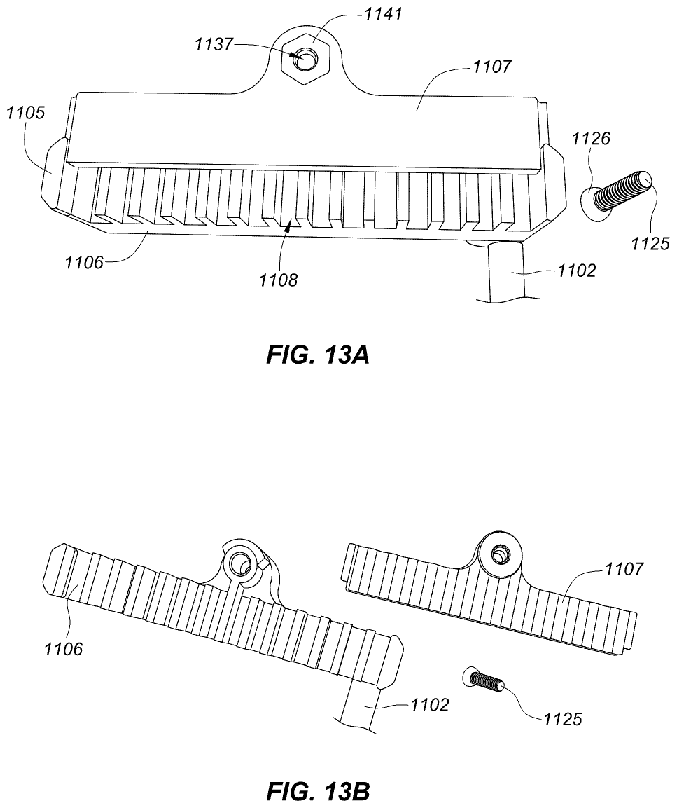

Referring particularly to FIGS. 13A and 13B, the first and second clasp components 1106 and 1107 can be separated from each other to insert device cables 1151 between the first and second clasp components 1106 and 1107. By employing multiple orifices 1108, the devices cables can be separated from each other. As an example, a screw 1125 is configured to removably couple the first clasp component 1106 to the second clasp component 1107. The screw 1125 includes a counter sunk head 1126 configured to be disposed in the first clasp component 1106 or the second clasp component 1107. At least one of the first or second clasp components 1106 and 1107 may include a threaded bore 1137, such as an embedded metal nut 1141 configure to receive the screw 1125.

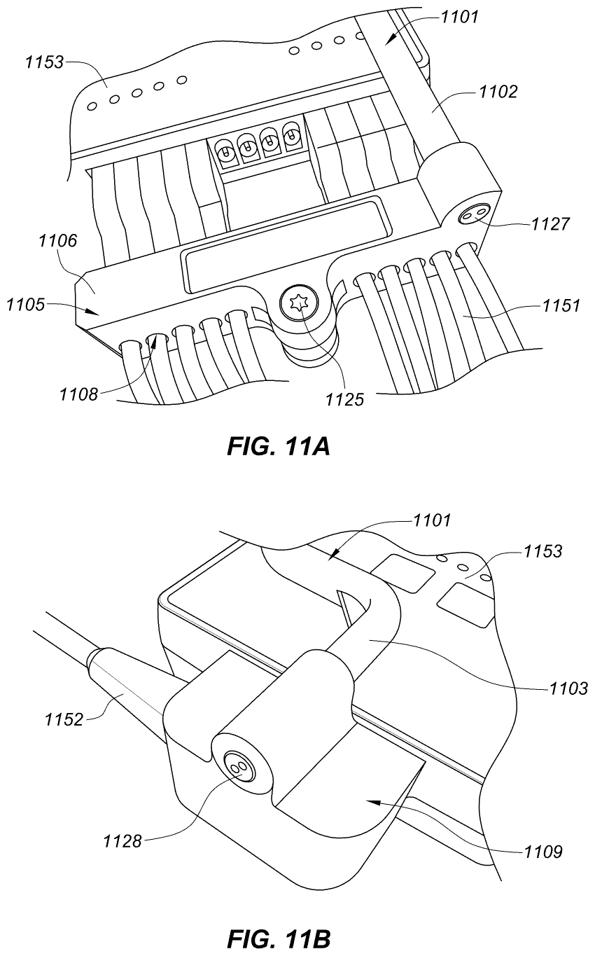

Referring particularly to FIG. 11A, a first screw 1127 is configured to couple the first clasp component 1106 with the security rod 1101. The first screw 1127 may be threaded into a threaded bore 1138 (see, e.g., FIG. 14A) formed in the first end portion 1102 of the security rod 1101. The first screw 1127 (e.g., a counter-sunk head of the first screw) is configured to be flush with a surface of the first clasp component 1106 facing away from the medical device 1153.

Referring particularly to FIG. 11B, a second screw 1128 is configured to couple the second locking clasp 1109 with the security rod 1101. The second screw 1128 may be threaded into a threaded bore 1139 (see, e.g., FIG. 14B) formed in the second end portion 1103 of the security rod 1101. The second screw 1128 (e.g., a counter-sunk head of the second screw) is configured to be flush with a surface of the second locking clasp 1109 facing away from the medical device 1153.

Referring particularly to FIG. 14A, a cuboidal segment 1111 is defined by the first end portion 1102 of the security rod 1101. A cuboidal orifice 1112 is formed in the first clasp component 1106 of the first locking clasp 1105. The cuboidal orifice 1112 is configured to receive the cuboidal segment 1111 to couple the first clasp component 1106 with the first end portion 1102 of the security rod 1101. The cuboidal segment 1111 of the first end portion 1102 of the security rod 1101 is configured to prevent rotation of the first locking clasp 1105.

Referring particularly to FIG. 14B, the second end portion 1103 of the security rod 1101 defines a cuboidal segment 1143 configured to prevent rotation of the second locking clasp 1109.

In an aspect of the present disclosure, the extension portion 1104 of the security rod 1101 defines a cylindrical shape. A first cross-sectional area defined by the cuboidal segment 1111 of the first end portion 1102 of the security rod 1101 is smaller than a second cross-sectional area defined by the extension portion 1104 of the security rod 1101.

In an aspect of the present disclosure, the extension portion 1104 of the security rod 1101 defines a cylindrical shape. A first diameter of the cuboidal segment 1111 of the first end portion 1102 of the security rod 1101 is smaller than a second diameter of the extension portion 1104 of the security rod 1101.

In an aspect of the present disclosure, the security rod 1101 is a rigid rod configured to prevent movement of the first locking clasp 1105 or the second locking clasp 1109 with respect to the medical device 1153.

In an aspect of the present disclosure, the security rod 1101 includes aluminum. For example, the security rod 1101 may include or may be formed of aluminum, such as uncoated aluminum. The security rod 1101 may be a single, integrally formed unit.

Referring particularly to FIG. 10, the first locking clasp 1105 and the second locking clasp 1109 extend from the security rod 1101 to align the first locking clasp 1105 with an upper surface of the medical device 1153 and to align the second locking clasp 1109 with a bottom surface of the medical device 1153. The first orifice(s) 1108 of the first locking clasp 1105 and the second orifice 1110 of the second locking clasp 1109 each extend in a plane offset from a plane in which the security rod 1101 extends.

Referring generally to FIGS. 10, 11B, 12B, and 14B, the second locking clasp 1109 includes a cradle 1129 configured to hold a connecting arm of the second device cable 1152. The second orifice 1110 extends from the cradle 1129.

In an aspect of the present disclosure, the cradle 1129 is configured to face the medical device 1153. The second orifice 1110 extends in a direction perpendicular to a direction in which the cradle 1129 faces.

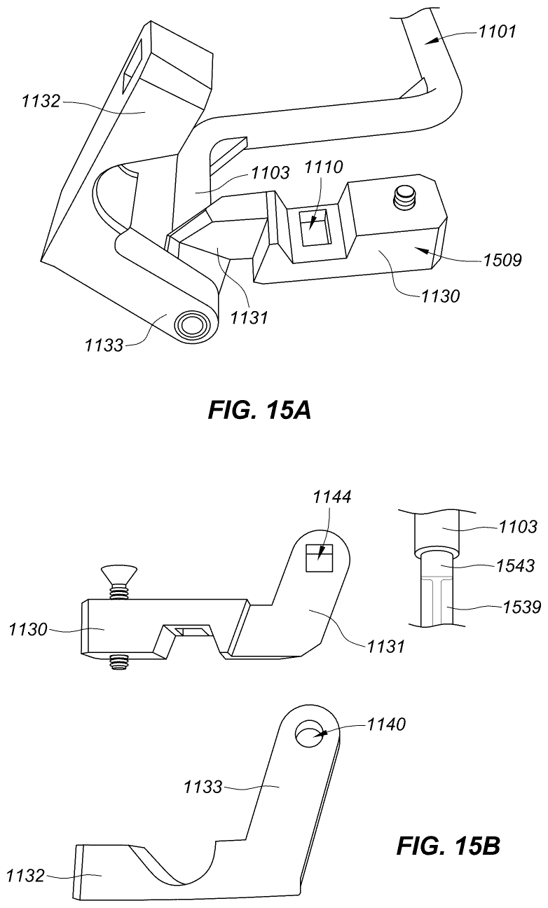

Referring particularly to FIGS. 15A and 15B, another second clasp component 1509 includes a third clasp component 1130 including a first security rod connecting arm 1131 connecting the third clasp component 1130 with the security rod 1101. A fourth clasp component 1132 includes a second security rod connecting arm 1133 connecting the fourth clasp component 1132 with the security rod 1101. A second orifice 1110 is defined between the third clasp component 1130 and the fourth clasp component 1132.

In an aspect of the present disclosure, a cuboidal segment 1543 is defined by the second end portion 1103 of the security rod 1101. A cuboidal orifice 1144 is defined in the first security rod connecting arm 1131. The cuboidal orifice 1144 is configured to receive the cuboidal segment 1543 to prevent rotation of the third clasp component 1130. A cylindrical segment 1539 is defined by the second end portion 1103 of the security rod 1101. A cylindrical orifice 1140 is defined in the second security rod connecting arm 1133. The cylindrical orifice 1140 is configured to receive the cylindrical segment 1539 to allow rotation of the fourth clasp component 1132 with respect to the third clasp component 1130. The order/arrangement of the cuboidal segment 1543 and the cylindrical segment 1539 may be reversed, and thus the arrangement of the corresponding connecting arms connecting thereto may similarly be reversed.

Various screws (e.g., connecting screws) are defined herein. Each of the screws described herein may include a counter-sunk screw head.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

1. A cable lock, comprising:
a security rod defining a first end portion, a second end portion, and an extension portion extending between the first end portion and the second end portion of the security rod;
a first locking clasp coupled with the first end portion of the security rod, wherein the first locking clasp includes:
a first clasp component coupled with the first end portion of the security rod; and
a second clasp component at least partially separable from the first clasp component, wherein at least one of the first clasp component or the second clasp component define at least one first orifice configured to receive at least one first device cable; and
a second locking clasp coupled with the second end portion of the security rod, wherein the second clasp component includes at least one second orifice configured to receive at least one second device cable,
wherein the security rod, the first locking clasp, and the second locking clasp are configured to prevent removal of the at least one first device cable and the at least one second device cable from a medical device.

2. The cable lock of claim 1, further including:
a cuboidal segment defined by the first end portion of the security rod; and
a cuboidal orifice formed in the first clasp component of the first locking clasp, wherein the cuboidal orifice is configured to receive the cuboidal segment therein to couple the first clasp component with the first end portion of the security rod, and wherein the cuboidal segment of the first end portion of the security rod is configured to prevent rotation of the first locking clasp.

3. The cable lock of claim 2, wherein the extension portion of the security rod defines a cylindrical shape, and wherein a first cross-sectional area defined by the cuboidal segment of the first end portion of the security rod is smaller than a second cross-sectional area defined by the extension portion of the security rod.

4. The cable lock of claim 2, wherein the extension portion of the security rod defines a cylindrical shape, and wherein a first diameter of the cuboidal segment of the first end portion of the security rod is smaller than a second diameter of the extension portion of the security rod.

5. The cable lock of claim 1, wherein the security rod is a rigid rod configured to prevent movement of the first locking clasp or the second locking clasp with respect to the medical device.

6. The cable lock of claim 1, wherein the security rod includes aluminum.

7. The cable lock of claim 1, wherein the extension portion of the security rod defines a first vertical portion, a second vertical portion, and a horizontal portion connecting the first vertical portion with the second vertical portion, wherein the first vertical portion, the second vertical portion, and the horizontal portion are arranged to allow the medical device to be positioned in a support sleeve.

8. The cable lock of claim 7, further including a first curved portion connecting the first vertical portion with the horizontal portion, and a second curved portion connecting the second vertical portion with the horizontal portion.

9. The cable lock of claim 1, wherein the first locking clasp and the second locking clasp extend from the security rod to align the first clasp component and the second clasp component with an upper surface of the medical device and to align the second locking clasp with a bottom surface of the medical device.

10. The cable lock of claim 9, wherein the at least one first orifice of the first locking clasp and the at least one second orifice of the second locking clasp each extend in a plane offset from a plane in which the security rod extends.

11. The cable lock of claim 1, further including at least one screw configured to removably couple the first clasp component to the second clasp component.

12. The cable lock of claim 11, wherein the screw includes a counter sunk head configured to be disposed in the first clasp component.

13. The cable lock of claim 1, further including a first screw configured to couple the first clasp component with the security rod, wherein the first screw is configured to be flush with a surface of the first clasp component facing away from the medical device.

14. The cable lock of claim 13, further including a second screw configured to couple the second clasp component with the security rod, wherein the second screw is configured to be flush with a surface of the second clasp component facing away from the medical device.

15. The cable lock of claim 1, wherein the second end portion of the security rod defines a cuboidal segment configured to prevent rotation of the second locking clasp.

16. The cable lock of claim 1, wherein the second clasp component includes:
a cradle configured to hold a connecting arm of the at least one second device cable therein, wherein the at least one second orifice extends from the cradle.

17. The cable lock of claim 16, wherein the cradle is configured to face the medical device, and wherein the at least one second orifice extends in a direction substantially perpendicular to a direction in which the cradle faces.

18. The cable lock of claim 1, wherein the second clasp component includes:
a third clasp component including a first security rod connecting arm connecting the third clasp component with the security rod; and
a fourth clasp component including a second security rod connecting arm connecting the fourth clasp component with the security rod,
wherein the at least one second orifice is defined between the third clasp component and the fourth clasp component.

19. The cable lock of claim 18, further including:
a cuboidal segment defined by the second end portion of the security rod;
a cuboidal orifice defined in the first security rod connecting arm, wherein the cuboidal orifice is configured to receive the cuboidal segment therein to prevent rotation of the third clasp component;
a cylindrical segment defined by the second end portion of the security rod; and
a cylindrical orifice defined in the second security rod connecting arm, wherein the cylindrical orifice is configured to receive the cylindrical segment therein to allow rotation of the fourth clasp component with respect to the third clasp component.

20. The cable lock of claim 19, further including a washer arranged about the cylindrical segment defined by the second end portion of the security rod, wherein the washer is configured to receive a screw therein connecting the second locking clasp with the security rod.
